(19) United States
(12) Patent Application Publication
(10) Pub. No.: US 2020/0353107 A1
(43) Pub. Date: Nov. 12, 2020

(54) IN VIVO IMAGING OF TUMOR INFILTRATION LEUKOCYTES

(71) Applicant: STC.UNM, Albuquerque, NM (US)

(72) Inventor: Jeffrey P. Norenberg, Albuquerque, NM (US)

(21) Appl. No.: 16/770,214

(22) PCT Filed: Dec. 7, 2018
(86) PCT No.: PCT/US2018/064485

§ 371 Date: Jun. 5, 2020

Related U.S. Application Data
(60) Provisional application No. 62/596,365, filed on Dec. 8, 2017.

(51) Int. Cl. A61K 51/04; A61K 45/06

(52) U.S. Cl. CPC A61K 51/0453; A61K 45/06

(57) ABSTRACT

The present invention is directed to the use of radiolabeled ligands of leukocyte function-associated antigen˜1 (LFA˜1) receptor in order to image and quantify leukocyte activation, recruitment and in vivo trafficking of tumor infiltrating lymphocytes. Diagnostic methods and methods of monitoring cancer therapy, including immunotherapy represent embodiments of the present invention.

### RELATED APPLICATIONS

This application claims the benefit of priority of U.S. provisional application Ser. No. 62/596,365 of identical title, filed Dec. 8, 2017, the entire contents of said application being incorporated by reference in its entirety herein.

### FIELD OF THE INVENTION

The present invention is directed to the use of radiolabeled ligands of leukocyte function-associated antigen-1 (LFA-1) receptor in order to image and quantify leukocyte activation, recruitment and in vivo trafficking of tumor infiltrating leukocytes and lymphocytes. Diagnostic methods and methods of monitoring cancer therapy represent embodiments of the present invention.

### BACKGROUND AND OVERVIEW OF THE INVENTION

The need for a non-invasive imaging of tumor infiltrating leukocytes is very important to both the diagnosis and treatment of cancer. There are currently no methods to non-invasively evaluate leukocyte/lymphocyte activation, recruitment, and trafficking to solid tumors. This novel application of R*-DANBIRT (radiolabeled DANBIRT) and related analogs allows quantification of leukocyte trafficking to tumors, in order to: characterize tumor infiltrating leukocytes to identify response: monitor response to immunotherapy; establish the stage of disease, including metastasis: detect residual disease after therapy; and to direct personalized medicine. The advantage of this non-invasive imaging radioligand is the sensitivity with which the radiopharmaceutical can detect the disease and its spread in the body.

Leukocyte function-associated antigen-1 (LFA-1) receptors are normally expressed by all white blood cells. A small molecule, alkylamino-NorBirt, previously developed as an allosteric inhibitor of LFA-1, has been functionalized through the addition of the chelator, 1,4,7,10-tetraazacyclododecane-N,N′,N″,N″′ tetraacetic acid (DOTA) to form DOTA-alkylamino-NorBirt or DANBIRT. The previous work of the inventors has demonstrated that a wide variety of polyvalent, cationic radiometals such as ⁶⁸Ga and ¹¹¹In, among numerous others as described herein can be effectively incorporated and radiolabeled to provide a radiolabeled LFA-1 ligand which is most preferably DANBIRT (R*-DANBIRT as the radiolabeled version). The inventors have shown that the resultant R*-DANBIRT retains its binding affinity towards LFA-1 on leukocytes/lymphocytes and can be used as an in vivo non-invasive PET/SPECT imaging agent to quantify LFA-1 receptor expression on lymphocytes and lymphocyte trafficking in vivo.

### BRIEF DESCRIPTION OF THE INVENTION

Leukocyte function-associated antigen-1 (LFA-1) receptor expression can be imaged using a radiolabeled LFA-1 ligand, preferably, R*-DANBIRT a novel radiolabeled small molecule to quantify leukocyte activation, recruitment, and in vivo trafficking of tumor infiltrating lymphocytes. It is noted that in instances where cancer tissue is present, the number and/or trafficking of leukocytes/lymphocytes tends to be diminished in that tissue compared to normal, healthy tissue.

The present invention relates to methods for imaging leukocytes and lymphocytes in order to non-invasively evaluate leukocyte activation, recruitment and trafficking to solid tumors. This novel method allows the characterization of tumor infiltrating leukocytes and/or lymphocytes in order diagnose the existence and extent of cancer and to identify response for diagnosis and/or therapy, including monitoring the response to immunotherapy, determining the stage and extent of the disease, to detect residual disease after therapy and to direct personalized medicine (for example, by monitoring therapy and establishing and/or changing the course of therapy to a patient who is not adequately responding to therapy). The advantage of this non-invasive imaging radioligand is the fact that the agent may be administered in vivo and the heightened binding these compounds exhibit to leukocytes and/or lymphocytes and heightened sensitivity with which the radiopharmaceutical can detect the disease (cancer) and its spread in the body of a patient. This approach is useful for all types of immunotherapy, including chimeric antigen receptor T-cell (CART) therapy, T-cell receptor therapy (TRT therapy), tumor-infiltrating lymphocytes (TIL therapy), monoclonal antibodies, immune checkpoint inhibitors and cancer-vaccines, among others, including general immunotherapies (e.g., interleukins, interferons, colony stimulating factors and agents which boost the immune system such as imiquimod (Zyclara), lenalidomide (Revlimid), pomalidomide (Pomalyst), and thalidomide) for numerous cancers, especially including solid tumors. In addition, the present methods may be used in the diagnosis and treatment of neuroinflammation, Alzheimer's disease, Parkinson's disease, Huntington's disease, amyotrophic lateral sclerosis (AML), motor neuron disease (MND), Creutzfeldt-Jacob disease, primary progressive aphasia, progressive supranuclear palsy and other neurodegenerative diseases, chronic pain (including chronic neuropathic pain and central and peripheral neuropathy) and fatigue disorders, and therapies to treat same by providing a method of diagnosing the type and extent of disease and monitoring therapy of these diseases and/or disorders and either maintaining a successful therapy or modifying a therapy in need of modification because of poor outcome or projected prognosis.

Thus, the present invention relates to the use of compounds according to the chemical structure:

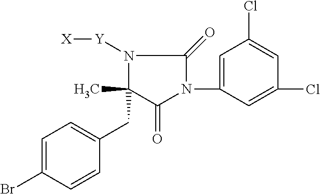

Where Y is a chemical linker which links the nitrogen to a chelate group or tricarbonyl complex X, wherein X incorporates or complexes with a radioisotope, R. In preferred aspects of the invention, Y is an optionally substituted hydrocarbyl (including an optionally substituted aryl group), preferably an optionally substituted alkyl group, for example a —(CH₂)ₙZ-group, where n is from 1 to 6 and Z is O, NR or N(R)—CH₂CH₂—O, where R is H or a C₁-C₃ alkyl (preferably H) or Z is a keto (C═O) group, a S(O)_{w} group where w is front 0 to 4 (i.e., a sulfide, sulfoxide, sulfone, sulfonate or sulfate group), a phosphonate group or a phosphate group and X is a chelate group in which a radioisotope is incorporated or complexed to diagnose cancer and/or the response of cancer, especially tumors, to therapy. In certain preferred aspects, Y is a —(CH₂)ₙNH-group, where n is from 1 to 6, preferably from 2 to 4, preferably 4 and X is a polyaminocarboxylic macrocycle, preferably 1,4,7,10-tetraazacyclododecane-1,4,7,10-tetraacetic acid (DOTA).

In other aspects of the invention, Y is a linker comprising a C₁-C₁₀, preferably a C₃-C₈ substituted hydrocarbyl group (which is bonded to the nitrogen of the dioxoimidazolyl group through a keto group) containing two amino groups or two sulfur groups which are linked with the tricarbonyl compound X which incorporates or complexes to the radioisotope. In certain aspects, the preferred linker contains a dithiahexyl group or a diaminohexyl or diaminobutyl group. In another aspect, the linker may be derived from lysine (linked to the dioxoimidazolinyl group through the carboxylic acid moiety of lysine). Chemical linkage of the linker to the dioxoimidazolinyl group may be through a carbonyl group, alkylene group or other group capable of being linked to the nitrogen of the dioxoimidazolinyl group.

R is a radioisotope, preferably a polyvalent cationic radioisotope, even more preferably a radioisotope selected from the group consisting of ⁸⁶Y, ⁹⁰Y, ¹¹¹In, ¹⁷⁷Lu, ²²⁵Ac, ²¹²Bi, ²¹³Bi, ⁶⁶Ga, ⁶⁷Ga, ⁶⁸Ga, ⁶⁴Cu, ⁶⁷Cu, ⁷¹As, ⁷²As, ⁷⁶As, ⁷⁷As, ⁶⁵Zn, ⁴⁸V, ²⁰³Pb, ²⁰⁹Pb, ²¹²Pb, ¹⁶⁶Ho, ¹⁴⁹Pm, ¹⁵³Sm, ²⁰¹Tl, ¹⁸⁸Re, ¹⁸⁶Re, and ^{99m}Tc. In certain preferred embodiments, the radioisotope is ⁶⁸Ga or ¹¹¹In as otherwise described herein.

Compounds according to the present invention exhibit a favorable bioavailability in vivo and a selectivity for binding to leukocytes and/or lymphocytes which are characterized by their ability to invade and traffic in tumors for the diagnosis of the existence and the extent of cancer in a patient by identifying the number of leukocytes and/or lymphocytes in tissue which binds to the above-identified ligand. It is unexpected that the methods according to the present invention are particularly useful for diagnosing the existence and progression of tumorous cancers and neurological disease states and conditions described herein.

In embodiments, the LFA-1 ligand is a compound in which X is a DOTA group, Y is a butyl amine group (such that the amine group of forms an amide group with one of the carboxylic acid groups of DOTA, linking DOTA to the LFA-1 binding moiety) to provide a compound according the general chemical structure:

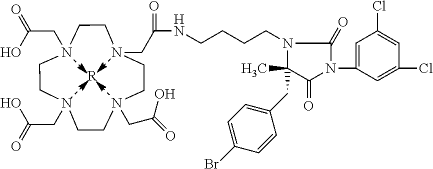

Where R is a radioisotope, preferably a cationic radioisotope, more preferably a polyvalent cationic radioisotope, or a pharmaceutically acceptable salt. It is noted that when the carboxylic acid group is in its carboxylate form (depending on the pH of the surrounding environment, e.g., at higher pH's), the carboxylate anion can complex with the radionuclide as shown below, depending on the valency of the radionuclide. In some instances, where the radionuclide is a quaternary polyvalent cation (4+), the carboxylate groups, as well as the carbonyl of the adjacent amide group may be complexed with the radionuclide. When the radionuclide is dicationic, one of the carboxylic acid groups remains in its carboxylate form and is uncomplexed to the radionuclide. FIG. 1 attached hereto shows different (but not all) forms of the same sub-generic compound which will complex carboxylate to the radionuclide depending upon the pH of the environment as well as the valency of the radionuclide.

In preferred embodiments of the invention the carboxylate anions of the DOTA group dictate to the radioisotope, wherein the LFA-1 ligand is a compound according to the chemical structure:

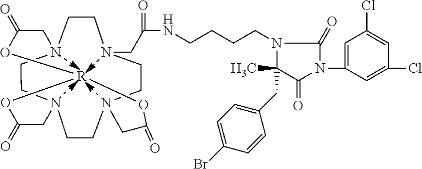

Where R is a radioisotope, preferably a polyvalent cationic radioisotope, even more preferably a radioisotope selected from the group consisting of ⁸⁶Y, ⁹⁰Y, ¹¹¹In, ¹⁷⁷Lu, ²²⁵Ac, ²¹²Bi, ²¹³Bi, ⁶⁶Ga, ⁶⁷Ga, ⁶⁸Ga, ⁶⁴Cu, ⁶⁷Cu, ⁷¹As, ⁷²As, ⁷⁶As, ⁷⁷As, ^{6S}Zn, ⁴⁸V, ²⁰³Pb, ²⁰⁹Pb, ²¹²Pb, ¹⁶⁶Ho, ¹⁴⁹Pm, ¹⁵³Sm, ²⁰¹Tl, ¹⁸⁸Re, ¹⁸⁶Re, and ^{99m}Tc. In certain preferred embodiments, the radioisotope is ²¹³Bi, ¹⁷⁷Lu, ⁶⁸Ga or ¹¹¹In, in other embodiments, the radioisotope is ⁶⁸Ga or ¹¹¹In. In certain embodiments, R is selected from the group consisting of ¹¹¹In, ⁶⁸Y, ⁴⁶Ga, ⁶⁷Ga, ⁶⁸Ga, ²⁰³Pb, ⁶⁴Cu and ^{99m}Tc when the compounds are to be used diagnostically or to monitor therapeutic intervention and R is selected from the group consisting of ⁹⁰Y, ¹⁷⁷Lu, ¹⁸⁶Re, ¹⁸⁸Re, ²¹²Bi/²¹²Pb, ²¹³Bi, ¹⁴⁹Pm, ¹⁶⁶Ho and ¹⁵³Sm when compounds according to the present invention are used in radiation therapy to treat tumors or other disease states and/or conditions.

Methods of diagnosing or monitoring the treatment of cancer therapy represent an additional embodiment of the present invention. In this method, an effective amount of one or more compounds according to the present invention is administered to a patient in need thereof to provide non-invasive imaging of tissue-infiltrating, or in the case of tumor cancer, preferably tumor infiltrating leukocytes and/or lymphocytes to gauge the existence and/or extent of disease (cancer or other disease as described herein), the existence of metastasis and/or the response of the cancerous tumor or other disease state or condition to therapy. Pursuant to this method, a compound according to the present invention is administered to a patient (preferably by administration directly into or adjacent to the tissue or tumor although other routes of administration may be used) and after a period of time to allow the compound to bind to leukocytes/lymphocytes in the patient, the bound leukocytes/lymphocytes are imaged using single photon emission computed tomography (SPECT) or positron emission tomography (PET) in order to determine the levels or concentration of leukocytes/lymphocytes in the cancer or other tissue and comparing the image obtained to a standard (e.g. the standard may be an image obtained from one or more healthy patient(s), one or more sick patients with the same disease state to be diagnosed and/or treated, or the same patient at different times such as at the start of therapy or at various times during therapy), wherein the determined levels indicate the existence and/or extent of disease or the effect of therapy on the disease state in the patient.

It is an unexpected discovery that by using a LFA-1 radioligand pursuant to the present invention which binds to leukocytes and/or lymphocytes which invade cancerous tumors and other tissues such as neuronal tissue, diagnosis of these tissues, including cancerous tumors and/or the extent of the disease, including tumor progression, especially including metastasis, and/or monitoring of therapy of cancerous tumors and other disease states and/or conditions may occur readily in vivo with great accuracy, making it far easier for the clinician to both diagnose cancerous tumors and other tissue such as neuronal (especially central nervous system tissue), monitor the treatment and actually treat tumorous cancers and other disease states and conditions such as neuroinflammation, Alzheimer's disease, Parkinson's disease, Huntington's disease, amyotrophic lateral sclerosis (AML), motor neuron disease (MND), Creutzfeldt-Jacob disease, primary progressive aphasia, progressive supranuclear palsy and other neurodegenerative diseases, chronic pain (including chronic neuropathic pain and central and peripheral neuropathy) and fatigue disorders, among others.

### BRIEF DESCRIPTION OF THE FIGURES

FIG. 1 attached hereto shows different (but not all) forms of the same preferred sub-generic compound which will complex carboxylate of the chelate group to the radionuclide depending upon the pH of the environment as well as valency of the radionuclide.

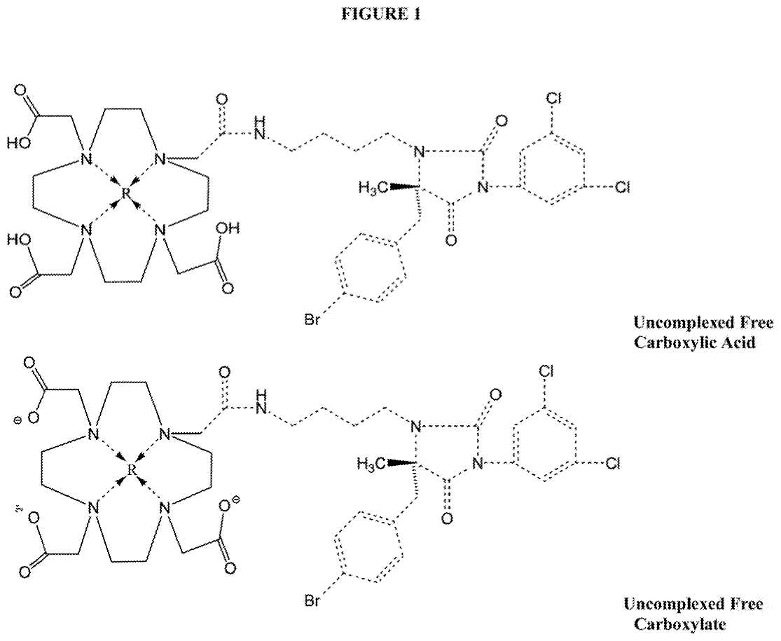

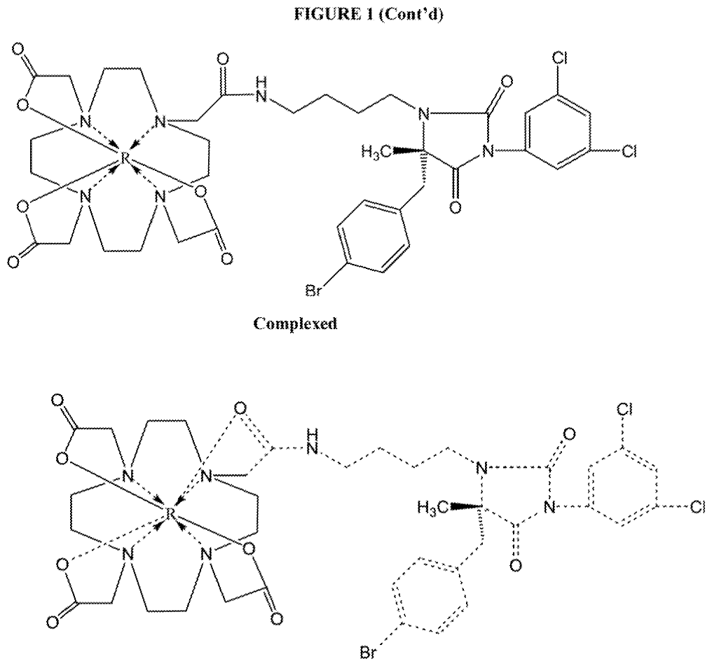

### DETAILED DESCRIPTION OF THE INVENTION

The following terms are used to describe the present invention. In instances where a term is not specifically defined herein, that term is given an art-recognized meaning by those of ordinary skill applying that term in context to its use in describing the present invention.

The term “compound”, as used herein, unless otherwise indicated, refers to any specific chemical compound disclosed herein and includes tautomers, regioisomers, geometric isomers, and where applicable, optical isomers (enantiomers) thereof, as well as pharmaceutically acceptable salts and derivatives (including prodrug forms) thereof. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds (at least about 70% enantiomerically enriched, preferably greater than 90% enantomerically enriched and in certain preferred embodiments, substantially pure or pure enantiomers where the compound is more than 98-99% or more enantiomerically enriched). The term also refers, in context, to prodrug forms of compounds which have been modified to facilitate the administration and delivery of compounds to a site of activity. It is noted that in describing the present compounds, numerous substituents, linkers and variables associated with same, among others, are described. It is understood by those of ordinary skill that molecules which are described herein are stable compounds as generally described hereunder and variables are chosen (often in combination) which promote the stability of the compound described.

The term “patient” or “subject” is used throughout the specification within context to describe an animal, generally a mammal and preferably a human, to whom treatment, including prophylactic treatment (prophylaxis), with the compositions according to the present invention is provided. For treatment of those infections, conditions or disease states which are specific for a specific animal such as a human patient or a patient of a particular gender, such as a human male or female patient, the term patient refers to that specific animal or that gender. Compounds according to the present invention are useful for the treatment, inhibition or prophylaxis (“reducing the likelihoods”) of cancer, including metastatic and recurrent cancer.

The term “effective” is used herein, unless otherwise indicated, to describe an amount of a compound or composition which, in context, is used to produce or effect an intended result, whether that result relates to the diagnosis, monitoring and/or the treatment of cancer, including metastatic cancer or the treatment of a subject for secondary conditions, disease states or manifestations of cancer as otherwise described herein. This term subsumes all other effective amount or effective concentration terms (including the term “therapeutically effective”) which are otherwise described in the present application.

The term “effective” is also used, to describe an amount of a compound, component or composition, which produces an intended effect when used within the context of its use, which may be a diagnostic method, a therapeutic method, a method to monitor the progression of therapy or other method (chemical synthesis) pursuant to the present invention. In the case of therapeutic methods, an effective amount for treating a tumor, including a metastatic tumor, is that amount which shrinks cancerous tissue (e.g., tumor), produces a remission, prevents further growth of the tumor and/or reduces the likelihood that the cancer in its early stages (in situ or invasive) does not progress further to metastatic melanoma.

Noted here is that within the context of the use of the present invention, the patient will be receiving a radiation dose, which provides guidance to the amount of compound which is considered effective when used within the context of its use. A patient undergoing a nuclear medicine procedure will receive a radiation dose. Under present international guidelines it is assumed that any radiation dose, however small, presents a risk. The radiation doses delivered to a patient in a nuclear medicine investigation present a very small risk of side effects, including inducing cancer in the patient. In this respect it is similar to the risk from X-ray investigations except that the dose is delivered internally rather than from an external source such as an X-ray machine.

The radiation dose from a diagnostic nuclear medicine procedure is expressed as an effective dose with units of sieverts (usually given in millisieverts, mSv). The effective dose resulting from an investigation is influenced by the amount of radioactivity administered in megabecquerels (MBq), the physical properties of the radiopharmaceutical used, its distribution in the body and its rate of clearance from the body.

Effective doses can range from 6 μSv (0.006 mSv) to 37 mSv or more for a 150 MBq thallium-201 non-specific tumour imaging procedure. The common bone scan with 600 MBq of technetium-99m-MDP has an effective dose of 3 mSv. Formerly, units of measurement w ere the Curie (Ci), being 3.7E10 Bq, and also 1.0 grams of radium (Ra-226); the rad (radiation absorbed dose), now replaced by the Gray; and the rem (röntgen equivalent man), now replaced with the Sievert. The rad and rem are essentially equivalent for almost all nuclear medicine procedures, and only alpha radiation will produce a higher Rem or Sv value, due to its much higher relative biological effectiveness (RBE).

The term “treating” or “successfully treating” when used within the context of treating a tumor, including a metastatic tumor, shall include shrinking a tumor, eliminating a tumor (resulting in a cure or remission), including a tumor which has metastasized (by causing a remission of the cancer in the patient) or reducing the likelihood or preventing the spread of the tumor into other organs. Tumors, including metastatic tumors, may be treated using compounds according to the present invention in combination, alone or in combination with other methods and/or compounds including surgery, chemotherapy, radiation therapy (i.e., with agents other than the present therapeutic compositions) and immunotherapy (IL-2 and/or α-interferon, among other immunotherapies as otherwise described herein).

The terms “treat”, “treating”, and “treatment”, etc., as used herein within context, also refers to any action providing it benefit to a patient at risk for cancer, especially a tumorous cancer, including the metastasis or recurrence of cancer, including improvement in the condition through lessening or suppression of at least one symptom, inhibition of cancer growth, reduction in cancer cells or tissue, prevention or delay in progression of metastasis of the cancer, prevention or delay in the onset of disease states or conditions which occur secondary to cancer or remission or cure of the cancer, among others. Treatment, as used herein, encompasses both prophylactic and therapeutic treatment. The term “prophylactic” when used, means to reduce the likelihood of an occurrence or the severity of an occurrence within the context of the treatment of cancer, including cancer metastasis as otherwise described hereinabove.

The term “leukocytes” refers to white blood cells in a patient's blood. The cellular components of blood include erythrocytes (red blood cells), leukocytes (white blood cells), and platelets. Normal human blood contains between about 4000-10,000 leukocytes/ μl. Leukocytes are divided into five classes based on morphological and tinetorial characteristics when stained. The five classes of leukocytes are:
neutrophils (40% -75%);
eosinophils (1% -6%);
basophils (less than 1%);
monocytes (2%-10%); and
lymphocytes (20% :45%)

Collectively, neutrophils, eosinophils, and basophils are known as granulocytes due to the presence of granules in their cytoplasm. In addition, monocytes and lymphocytes are also known as mononuclear cells.

The term “Lymphocytes” refers to a subset of white blood cells or leukocytes. Lymphocytes represent about 20% to about 45%. A lymphocyte is a type of white blood cells that is part of the immune system. Two main types of lymphocytes are B-cells and T-cells. B-cells are characterized by the presence of immunoglobulins on their surface, and upon stimulation with antigen, they are transformed into plasma cells. Plasma cells are then able to secrete antibodies specific to the antigen. T-cells take part in cell mediated immune response, which does not depend on the presence of circulating antibodies. T cells destroy the body's own cells that have themselves been taken over by viruses or become cancerous. Lymphocyte number are relevant to diagnosis of cancer and may be unregulated (increased compared to normal) or downregulated (reduced compared to normal) depending, upon the type of cancer or the stage of cancer which is diagnosed. Early stage cancer tends to have higher lymphocyte numbers compared to later stage cancers, which show reduced lymphocyte activity.

The term “tumor” is used to describe a malignant or benign growth or tumefacent.

The term “neoplasia” refers to the uncontrolled and progressive multiplication of tumor cells, under conditions that would not elicit, or would cause cessation of, multiplication of normal cells. Neoplasia results in a “neoplasm”, which is defined herein to mean any new and abnormal growth, particularly a new growth of tissue, in which the growth of cells is uncontrolled and progressive. Thus, neoplasia includes “cancer”, which herein refers to a proliferation of tumor cells having the unique trait of loss of normal controls, resulting in unregulated growth, lack of differentiation, local tissue invasion, and/or metastasis. The cancer may be “naïve”, metastatic or recurrent and includes drug resistant and multiple drug resistant cancers, all of which may be treated using compounds according to the present invention.

As used herein, neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. It is particularly unexpected that the present methods may be used so effectively to diagnose and/or monitor therapy in cancerous tumors. Malignant neoplasms are distinguished from benign neoplasms in that the former show a greater degree of anaplasia, or loss of differentiation and orientation of cells, and have the properties of invasion and metastasis. Examples of neoplasms or neoplasias from which the target cell of the present invention may be derived include, without limitation, carcinomas (e.g., squamous-cell carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, and stomach; benign and malignant lymphomas, particularly Burkitt's lymphoma and Non-Hodgkin's lymphoma; benign and malignant melanomas; myeloproliferative diseases; leukemias, sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas), germ-line tumors (e.g., bowel cancer, breast cancer, prostate cancer, cervical cancer, uterine/endometrial cancer, lung cancer, ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin; such as Wilms' tumor and teratocarcinomas, which may be treated by one or more compounds according to the present invention. See, (Beers and Berkow (eds.), The Merck Manual of Diagnosis and Therapy, 17.sup.th ed. (Whitehouse Station, N.J.: Merck Research Laboratories, 999) 973-74, 976, 986, 988, 991.

In certain particular aspects of the present invention, the cancer to which the present invention is applied, from a diagnostic and/or treatment perspective, is metastatic cancer. Metastatic cancer may be found in virtually all tissues of a cancer patient in late stages of the disease, including the lymph system/nodes (lymphoma)) in bones, in bladder tissue, in kidney tissue, liver tissue and in virtually any tissue, including brain (brain cancer/tumors). Thus, the present invention is generally applicable and may be used to treat any cancer in any tissue, regardless of etiology. In other instances, the cancer which is treated, including prophylactically treated, is a recurrent cancer, which often recurs after an initial remission. The present compounds also may be used to reduce the likelihood of a cancer recurring and for treating a cancer which has recurred.

The term “pharmaceutically acceptable salt” or “salt” is used throughout the specification to describe a salt form of one or more of the compositions herein which are presented to increase the solubility of the compound in saline for parenteral delivery or in the gastric juices of the patient's gastrointestinal tract in order to promote dissolution and the bioavailability of the compounds. Pharmaceutically acceptable salts include those derived from pharmaceutically acceptable inorganic or organic bases and acids. Suitable salts include those derived from alkali metals such as potassium and sodium, alkaline earth metals such as calcium, magnesium and ammonium salts, among numerous other acids well known in the pharmaceutical art. Sodium and potassium salts may be preferred as neutralization salts of carboxylic acids and free acid phosphate containing compositions according to the present invention. The term “salt” shall mean any salt consistent with the use of the compounds according to the present invention. In the case where the compounds are used in pharmaceutical indications, including the treatment of prostate cancer, including metastatic prostate cancer, the term “salt” shall mean a pharmaceutically acceptable salt, consistent with the use of the compounds as pharmaceutical agents.

The term “coadministration” shall mean that at least two compounds or compositions are administered to the patient at the same time, such that effective amounts or concentrations of each of the two or more compounds may be found in the patient at a given point in time. Although compounds according to the present invention may be co-administered to a patient at the same time, the term embraces both administration of two or more agents at the same time or at different times, provided that effective concentrations of all coadministered compounds or compositions are found in the subject at a given time. Compounds according to the present invention may be administered with one or more anti-cancer agents or other agents which are used to treat or ameliorate the symptoms of cancer. In the present invention, LFA-1 ligands may be used to diagnose and/or determine the response of a cancer to cancer therapy, often in conjunction with anticancer agents or alternative cancer therapies, such as radiation therapy, surgery, hormone therapy, immunotherapy, targeted therapy, heat or oxygenation therapy.

The term “anticancer agent” “additional anticancer agent” refers to a compound other than the chimeric compounds according to the present invention which may be used in combination with a compound according to the present invention for the treatment of cancer. Exemplary anticancer agents which may be coadministered in combination with one or more chimeric compounds according to the present invention include, for example, antimetabolites, inhibitors of topoisomerase I and II, alkylating agents and microtubule inhibitors (e.g., taxol), among others. Exemplary anticancer compounds for use in the present invention may include everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bel-2 inhibitor, an HDAC inhibitor, a c-M ET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab (Arzerra), zanolimumab, edotecarin, tetrandrine, rubitecan, tesmilifene, oblimersen, ticilimumab, ipilimumab, gossypol, Bio 111, 131-1-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr311, romidepsin, ADS-100380, sunitinib, 5-fluorouracil, vorinostat, etoposide, gemcitabine, doxorubicin, irinotecan, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1 H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, lentrozole, DES(diethylstilbestrol), estradiol, estrogen, conjugated estrogen, bevacizumab, IMC-1C11, CHIR-258,); 3-[5-(methylsulfonylpiperadinemethyl)-indolyl]-quinolone, vatalanib, AG-013736, AVE-0005, the acetate salt of [D-Ser(Bu t) 6,Azgly 10] (pyro-Glu-His-Trp-Ser-Tyr-D-Ser(Bu t)-Leu-Arg-Pro-Azgly-NH₂ acetate [C₅₉H₈₄N₁₈Oi₄-(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAR-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SUM248, sorafenib, KRN951, aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cvproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12,IM862, angiostatin, vitaxin, droloxifene, idoxyfene, spironolactone, finasteride, cimitidine, trastuzumab, denileukin diftitox, gefitinib, bortezimib, paclitaxel, irinotecan, topotecan, doxorubicin, docetaxel, vinorelbine, bevacizumab (monoclonal antibody) and erbitux, cremophor-free paclitaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, PTK787/ZK222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a. pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa, among others. Other anticancer agents which may be used in combination include immunotherapies such ipilimumab, pembrolizumab, nivolumab, alemtuzumab, atezolizumab, ofatumumab, novolumab, pembrolizumab, and rituximab, among others.

The term “imaging”, “molecular imaging” or “radioimaging” is used to describe methods that use the nuclear properties of matter in diagnosis and therapy, pursuant to the present invention. More specifically, the present invention relies on molecular imaging because it produces images that reflect biological processes that take place at the cellular and subcellular level.

Molecular imaging is a discipline that unites molecular biology and in vivo imaging. It enables the visualisation of the cellular function and the follow-up of the molecular process in living organisms without perturbing them. The multiple and numerous potentialities of this field are applicable to the diagnosis and treatment of diseases such as cancer, in the present invention, in particular, melanoma, including metastatic melanoma. This technique also contributes to improving the treatment of these disorders by optimizing the pre-clinical and clinical tests of new medication. This approach also has a major economic impact due to earlier and more precise diagnosis.

Molecular imaging differs from traditional imaging in that probes labeled biomarkers are used to help image particular targets or pathways. Biomarkers interact chemically with their surroundings and in turn alter the image according to molecular changes occurring within the area of interest. This process is markedly different from previous methods of imaging which primarily imaged differences in qualities such as density or water content. This ability to image fine molecular changes opens up an incredible number of exciting possibilities for medical application, including early detection and treatment of disease, in particular, melanoma and metastatic melanoma according to the present invention.

There are a number of different imaging modalities that can be used for noninvasive molecular imaging, using compounds according to the present invention. Each has different strengths and weaknesses and some are more adept at imaging multiple targets or sites than others. This is important in instances where metastatic melanoma is suspected. The modalities which can be used in the present invention are varied and in the present invention principally include single photon emission computed tomography (SPECT) and positron emission tomography (PET), discussed below.

The main purpose of SPECT when used in melanoma imaging pursuant to the present invention is to measure the distribution of radioisotope in skin tissue, in particular, those skin regions and other tissues where melanoma, including metastatic melanoma, is suspected. The development of computed tomography in the 1970s allowed mapping of the distribution of the radioisotopes in tissue, and led to the technique now called SPECT.

The imaging agent used in SPECT emits gamma rays, as opposed to the positron emitters used in PET. There are a number of radioisotopes (such as ^{99m}Tc, ¹¹¹In, ¹²³I, ²⁰¹Tl, ⁶⁷Ga, ^{99m}Tc and ²⁰³Pb, among other gamma ray emitters) that can be used in the present invention and imaged with SPECT technology. In SPECT, where possible, by rotating the gamma camera around the area to be analysed, a three dimensional image of the distribution of the radiotracer may be obtained by employing filtered back projection or other tomographic techniques. The radioisotopes used in SPECT have relatively long half lives (a few hours to a few days) making them easy to produce and relatively cheap in comparison to other radioisotopes. This represents the major advantage of SPECT as an imaging technique, since it is significantly cheaper than PET or other imaging methods such as magnetic resonance imaging (MRI). However, SPECT sometimes lacks exceptional spatial (i.e., where exactly the particle is) or temporal (i.e., did the contrast agent signal happen at a particular millisecond or not) resolution.

Another imaging technique which finds particular use in the present invention is positron emission tomography (PET). In PET, a molecule is tagged with a positron emitting isotope. These positrons (β particles) interact with nearby electrons, emitting two 511,000 eV photons, directed 180 degrees apart in opposite directions. These photons are then detected by the scanner which can estimate the density of positron annihilations in a specific area. When enough interactions and annihilations have occurred, the density of the original molecule may be measured in that area. Typical isotopes include ¹¹C, ¹³N, ¹⁵O, ¹⁸F, ⁶⁴Cu, ⁶²Cu, ¹²⁴I, ⁷⁶Br, ⁸²Rb and ⁶⁸Ga, among others, including the preferred ⁶⁶Ga, ⁶⁸Ga, ⁶⁴Cu, ⁸⁶Y. One of the major disadvantages of PET is that most of the radioisotopes must be made with a cyclotron, thus making the use of PET, in certain instances prohibitively expensive. Most of these probes also have a half life measured in minutes and hours, thus forcing the cyclotron, in many instances, to be on site. These factors can make PET sometimes prohibitively expensive, except in certain cases, which the present invention addresses in certain aspects. PET imaging does have many advantages though. First and foremost is its sensitivity: a typical PET scanner can detect between 10⁻¹¹ mol/L to 10⁻¹² mol/L concentrations.

These and other imaging approaches are well known in the art. See, for example, Wei, et al., Trends in Cancer, Vol. 4, No. 5, pp. 359-373 (2018) and van der Veen, et al., Cancer Treatment Reviews, 70, 232-244 (2018), each of which is incorporated by reference herein.

In one embodiment, administration of compounds/compositions according to the present invention assist in monitoring therapies for treating or curing cancer wherein during treatment of cancer, a compound according to the present invention may be administered (by any route of administration, but preferably by intravenous administration) to a patient such that cancer tissue may be imaged/monitored and optionally/preferably compared to a standard image (from uninfected tissue and/or infected tissue including tissue from the patient at the commencement of treatment) in order to determine the effect of therapy on the diseased tissue. The therapy may thereafter be terminated because a cure has been effected, the same therapy may be continued to further treat the infection, or the therapy may be modified in order to further treat the infection based upon the results of imaging.

Preparation of compounds according to the present invention proceeds using standard synthetic chemical techniques which are readily available in the art. Synthetic methods for obtaining compounds related to the present invention may be found in U.S. Pat. No. 6,881.747, issued Apr. 19, 2005, which is incorporated by reference herein. These methods can serve as guides for obtaining compounds according to the present invention. In general, the present compounds may be made by condensing a chelate compound to which is bound a radionuclide onto an activated moiety containing either an electrophilic group or a nucleophilic group of a linker group which is chemically linked to the amine of the dioxoimidazolidine group of the compounds according to the present invention. Alternatively, the chelate may be first reacted with one end of a difunctional chemical linker and the unreacted moiety of the linker group may thereafter be reacted with the dioxoimidazoline group. Radioisotopes may be added (chelated) to the compound at an early or later stage in the chemical synthetic method by methods routine in the art.

As discussed above, tricarbonyl complexes may be used to prepare the final diagnostic/therapeutic compound according to the present invention. Preparation of the compound can also be prepared using Technetium (I) and Rhenium (I) tricarbonyl complexes such as those listed below using methods described by H.-J. Pietzsch, A. Gupta, M. Reisgys, A. Drews, S. Seifert, S. Seifert, et. al. [Chemical and Biological Characterization of Technetium(I) and Rhenium(I) Tricarbonyl Complexes with Dithioether Ligands Serving as Linkers for Coupling the Tc(CO)₃ and Re(CO)₃ Moieties to Biologically Active Molecules, Bioconjugate Chem., 11(3) 414-424, 2000].
Bromo(3,6-dithiaoetane-S,S)tricarbonylrhenium(I)]
[Bromo(4,7-dithia-1-octyne-S,S)tricarbonylrhenium(I)]
[Bromo(1-carboxy-3,6-dithiaheptane-S,S)tricarbonylrhenium(I)] (C₉H₁₂BrO₅ReS₂)
[Bromo(1,6-dicarboxy-2,5-dithiahexane-S,S)tricarbonylrhenium(I)] (C₉H₁₀BrO₇ReS₂)
[(1-Carboxylato-3,6-dithiaheptane-O,S,S)tricarbonylrhenium(I)](C₉H₁₁O₅ReS₂)
[(1-Carboxylato-6-carboxy-2,5-dithiahexane-O,S,S)tricarbonylrhenium(I)] (C₉H₉O₇ReS₂)
[Bromo(1,8-dihydroxy-3,6-dithiaoctane-S,S)tricarbonylrhenium(I)] (C₉H₁₄BrO₅ReS₂)
[(1,8-Dihydroxy-3,6-diihiaoctane-O,S,S)tricarbonylrhenium(I)]nitrate (C₉H₁₄NO₈ReS₂)
[Chloro(3,6-dithiaoctane-S,S)tricarbonyltechnetium(I)]
[Chloro(4,7-dithia-1-octyne-S,S)tricarbonyltechnetium(I)]
[Chloro(1-carboxy-3,6-dithiaheptane-S,S)tricarbonyltechnetium(I)]
[Chloro(1,6-dicarboxy-2,5-dithiahexane-S,S)tricarbonyltechnetium(I)]
[1-Carboxylato-3,6-dithiaheptane-O,S,S)tricarbonyltechnetium(I)
[(1-Carboxylato-6-carboxy-2,5-dithialhexane-O,S,S)tricarbonyltechnetium(I)]

The tricarbonyl complexes as described above may be reacted with the dioxoimidazoinyl compound to form a chemically linked tricarbonyl complex which contains the radioisotope.

Attachment of metal radioisotopes to the compounds prepared above make the final NorBit diagnostic/therapeutic compounds. Analogous preparations yield compounds containing other radioisotopes as otherwise disclosed herein.

### Linkers

The linkers which may be used in the present invention are comprised of alkyl chains of various lengths and containing various side chains (optionally substituted) depending on the hydrophobic/hydrophilic properties of the final product and the clinical needs. Linkers preferably contain O, S or NH or other functional group on the distal end of the molecule in order to attach a chelate to which may be bound a radioisotope. Simple condensation or other reactions may be used to covalently link the linker to the chelate so that a radionuclide may be complexed accordingly.

The term “chelate”, “chelator” or “chelating agent” is used to describe a moiety (as represented by Y in generic structures) which is functionally capable of complexing or “chelating” a radioisotope as otherwise described herein. Each is appropriately chemically linked (via covalent linkers or directly to Cyclic peptides as otherwise described herein). Exemplary chelators for use in the present invention, which are well known in the art, include the following:
Polyaminocarboxylates, such as
EDTA: ethylenediaminetetraacetic acid
DTPA: diethylenetriaminepentaacetic acid
Polyaminocarboxylic Macrocycles, such as:
DOTA: 1,4,7.10-tetraazacyclododecane-1,4.7,10-tetraacetic acid
TRITA: 1,4,7,10-tetraazacyclotridecane-1,4,7,10-tetraacetic acid
TETA: triethylenetetramine bridged-cyclam-2a: 1,4,8,11-tetraazabicyclo[6.6.2]hexadecane-1,8- di(methanephosphonic acid)
DO3A: 1,4,7-tris(carboxymethyl)-1,4,7,10-tetraazacyclododecane
DO2A: 1,4,7,10-tetraazacyclododecane-1,7-bis(acetic acid )
Other Chelators, such as:
CB-TE2A (4,11-bis(carboxymethyl)-1,4,8,11-tetraazabicyclo[6,6.2]hexadecane)
NOTA (1,4,7-triazacyclononane-1,4,7-triacetic acid)
MAG₃ (Mercaptoacetyltriglycine)
4,5-bis(2-mercaptoacetamido)pentanoic acid.

Chelates, chelators or chelating agents are generally bi- or multidentate ligands which generally produce a binding or complexation (complex) of a metal radioisotope as otherwise described herein. The ligand or chelator forms a chelate complex with the substrate. The term, without limitation, is used to describe complexes in which the metal ion is bound to two or more atoms of the chelating agent by whatever means (e.g., coordinate binding or complexation) occurs when a radioisotope and chelate group complex within each other in compounds according to the present invention. It is noted here that when a chelator is complexed to a radioisotope as used herein, the chelate complex structure is represented in a generic, nonlimiting sense, such that bonds which are represented may occur between a radioisfope and the chelating agent, as well as additional bonds (such as between carbonyl/carboxyl groups) which are not specifically represented, but which are understood/determined to be bonded within the context of the chelate complex (to accommodate that different radioisotopes may bind differently to different chelate groups).

The term “DOTA” is used as an abbreviation for 1,4,7,10-tetraazacyclododecane-1,4,7,10-tetraacetic acid, a preferred chelator for use in the present invention, which chemical structure (bonded in compounds according to the present invention) is represented as follows:

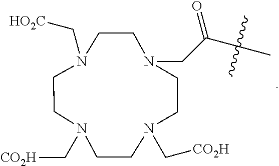 [SMILES: *C(=O)CN1CCN(CC(=O)O)CCN(CC(=O)O)CCN(CC(=O)O)CC1]

Complexed with radioisotopes according to the present invention, DOTA has the general chemical structure as described above (note that this general structure also includes the possibility of carbonyl/carboxyl groups also contributing to the complex depending on the radioisotope).

The term “standard” is used to describe a set of reference measurement(s) (which term includes a single measurement) made with for example, normal or non-diseased tissue (or, in some cases diseased and/or non-treated tissue) such that a comparison with a tested sample or samples can be made to determine the existence or absence of a disease-state or condition in the tested sample (which is usually in the patient's body) or the effectiveness of a therapeutic treatment on the response of the cancer, including remission. In the present invention, standards may be determined by taking measurements using normal tissue and/or the absence of a condition or disease state or a measurement, among other methods, for which the diagnostic assay is used. Standards are well known in the art and are determined using well known methods available in the art. Standards may vary from application to application depending upon the diagnostic method utilized.

The term “immunotherapy” refers to therapies which utilize the immune system of a patient or subject to treat a disease state or condition, especially a cancer. Various examples of immunotherapy are known in the art and include, for example, chimeric antigen receptor T-cell (CART) therapy, T-cell receptor therapy (TRT therapy), tumor-infiltrating lymphocyte therapy (TIL therapy), monoclonal antibodies, immune checkpoint inhibitors and cancer vaccines, among others, including general immunotherapies (e.g., interleukins, interferons, colony stimulating factors and agents which boost the immune system such as imiquimod (Zyclara), lenalidomide (Revlimid), pomalidomide (Pomalyst), and thalidomide). All of these therapies are known in the art and can be used with the methods of the present invention to monitor, assess the effectiveness of the therapy and to modify the therapy within the context of treatment to increase the likelihood of a favorable outcome for the patient or subject. A recent paper which indicates how molecular imaging may be used to elucidate and/or enlighten cancer immunotherapies and underlying processes is the review by van der Veen, et al., Cancer Treatment Reviews, 70, 232-244 (2018), which is incorporated by reference herein.

The present invention is also directed to pharmaceutical compositions comprising an effective amount of a compound according to the present invention, including the pharmaceutically acceptable acid or base addition salts of compounds of the present invention, optionally in combination with a pharmaceutically acceptable carrier, additive or excipient. These compounds may be used alone or in combination with other bioactive agents, especially including anticancer agents or anticancer therapies, or immunotherapy agents which are useful for treating or monitoring the treatment of and treating any one or more of the disease states which are described herein.

The compounds of formula I may, in accordance with the invention, be administered in single or divided doses by the oral, parenteral or topical routes. Administration of the active compound may range from continuous (intravenous drip) to several oral administrations per day (for example, Q.I.D.) and may include oral, topical, parenteral, intramuscular, intravenous, sub-cutaneous, transdermal (which may include a penetration enhancement agent), buccal and suppository administration, among other routes of administration. Parenteral, especially IV routes of administration are preferred for diagnostic methods. Enteric coated oral tablets may also be used to enhance bioavailability of the compounds from an oral route of administration. The most effective dosage form will depend upon the pharmacokinetics of the particular agent chosen as well as the severity of disease in the patient. Administration of compounds according to the present invention as sprays, mists, or aerosols for intra-nasal, intra-tracheal or pulmonary administration may also be used. The present invention therefore also is directed to pharmaceutical compositions comprising an effective amount of a compound according to the present invention, optionally in combination with a pharmaceutically acceptable carrier, additive or excipient,

The amount used is that amount effective within the context of the administration. A suitable oral dosage for a compound according to the present invention would be in the range of about 0.01 mg to 10 g or more per day, preferably about 0.1 mg to about 1 g per day. In parenteral formulations, a suitable dosage unit may contain from 0.1 to 250 mg of said compounds, which may be administered from one to four times per day, whereas for topical administration, formulations containing 0.01 to 1% active ingredient are preferred. It should be understood, however, that the dosage administration from patient to patient will vary and the dosage for any particular patient will depend upon the clinician's judgment, who will use as criteria for fixing a proper dosage the size and condition of the patient as well as the patient's response to the drug.

When the compounds of the present Invention are to be administered by the oral route, they may be administered as medicaments in the form of pharmaceutical preparations which contain them in association with a compatible pharmaceutical carrier, additive or excipient material. Such carrier material can be an inert organic or inorganic carrier material suitable for oral administration. Examples of such carrier materials are water, gelatin, talc, starch, magnesium stearate, gum arabic, vegetable oils, polyalkylene-glycols, petroleum jelly and the like.

The pharmaceutical preparations can be prepared in a conventional manner and finished dosage forms can be solid dosage forms, for example, tablets, dragees, capsules, and the like, or liquid dosage forms, for example solutions, suspensions, emulsions and the like.

The pharmaceutical preparations may be subjected to conventional pharmaceutical operations such as sterilization. Further, the pharmaceutical preparations may contain conventional adjuvants such as preservatives, stabilizers, emulsifiers, flavor-improvers, wetting agents, buffers, salts for varying the osmotic pressure and the like. Solid earlier material which can be used include, for example, starch, lactose, mannitol, methyl cellulose, microcrystalline cellulose, talc, silica, dibasic calcium phosphate, and high molecular weight polymers (such as polyethylene glycol).

For parenteral use, a compound according to the present invention can be administered in an aqueous or non-aqueous solution, suspension or emulsion in a pharmaceutically acceptable oil or a mixture of liquids, which may contain bacteriostatic agents, antioxidants, preservatives, buffers or other solutes to render the solution isotonic with the blood, thickening agents, suspending agents or other pharmaceutically acceptable additives. Additives of this type include, for example, tartrate, citrate and acetate buffers, ethanol, propylene glycol, polyethylene glycol, complex formers (such as EDTA), antioxidants (such as sodium bisulfite, sodium metabisulfite, and ascorbic acid), high molecular weight polymers (such as liquid polyethylene oxides) for viscosity regulation and polyethylene derivatives of sorbitol anhydrides. Preservatives may also be added if necessary, such as benzoic acid, methyl or propyl paraben, benzalkonium chloride and other quaternary ammonium compounds.

The compounds of this invention may also be administered as solutions for nasal application and may contain in addition to the compounds of this invention suitable buffers, tonicity adjusters, microbial preservatives, antioxidants and viscosity-increasing agents in an aqueous vehicle. Examples of agents used to increase viscosity are polyvinyl alcohol, cellulose derivatives, polyvinyipyrrolidone, polysorbates or glycerin. Preservatives added may include benzalkonium chloride, chloro-butanol or phenylethyl alcohol, among numerous others.

Additionally, the compounds provided by the invention can be administered by suppository.

In certain aspects according to the present invention, where various cancers are to be treated, the compounds may be co-administered with at least one other anti-cancer agent as otherwise described herein.

### EXAMPLES

### Research Methods

Radiolabelling the DOTA-alkylamino-NorBirt with radionuclide and determining its specific activity, specific binding and integrity towards LFA-1 receptors on leukocytes and/or lymphocytes utilizing in vitro receptor studies may be performed according to the methods which are described in detail in U.S. patent application No. 2007004826, which is incorporated by reference herein.

### Objective

To evaluate the in vivo molecular imaging potential of this compound in a pre-clinical model of tumor diagnosis and monitoring of cancer therapy.

### Methods

### Radiolabeling

The radiometal ¹¹¹In is incorporated into alkylamino-NorBIRT through 1,4,7,10-tetraazacyclododecane-N,N′N″N′″-tetraacetic acid (DOTA) as a chelator. ¹¹¹In chloride (high purity) was purchased through Mallinekrodt (United States). Synthesis of the alkylamino-NorBIRT is described in detail in Cancer Biotherapy and Radiopharmaceuticals Volume 21, Number 5, 2006, pages 418-426.

DOTA-butylamino-NorBIRT is dissolved in ultrapure water. ¹¹¹In-chloride is placed in a metal free tube and the NorBirt solution is added. The solution is mixed and then buffered to a pH of 5-6 using a 3M ammonium acetate buffer. The solution is heated in a hot block for 30 minutes at 100° C. The reaction mixture (50 uL) is added to 200 uL of 4 mM diethylenetriaminepentaacetic acid (DTPA, Mallinekrodt Baker Inc., Paris, Ky.).

Incorporation yield is determined using ITLC silica gel strips (Gelman Sciences, Inc., Ann Arbor, Mich.) with 0.9% NaCl USP solution (Hospira Inc., Lake Forest, Ill.). Stripes are analyzed on an AR2000 (Bioscan Inc., Washington, D.C.).

### Redistribution

An initial biodistribution study is carried out in laboratory test animals at 5 hours post-injection of ¹¹¹In-DOTA-alkylamino-NorBIRT. Results are evaluated as the percent injected dose per gram of tissue. The organs assessed are the heart, blood, stomach, liver, spleen, adrenals, kidneys, bone, muscle, bladder, testes, as well as the abcess or site of infection.

### Imaging

Mice are imaged with the Bioscan NanoSPECT/CT imaging system. Dynamic images are obtained immediately following injection of ˜750 uCi of ¹¹¹In-ROTA-alkylamino-NorBIRT intravenously. Static images are also obtained at 2, 4, and 24 hours post injection. Images are individually characterized.

### Results

ITLC analyses of ¹¹¹In-DOTA-alkylamino-NorBIRT demonstrates ≥98% incorporation yield. The specific activity is 473 Ci/mmol. SPECT/CT images with ¹¹¹In-alkylaminoNorBIRT show focal uptake in the tumor, and prompt and significant urinary excretion as soon as 5 minutes post-injection and at all subsequent time points.

### Discussion

The radiometal ¹¹¹In is a polyvalent cationic metal that is an ideal candidate for SPECT imaging with 173 and 245 keV energy peaks. Gallium-68 is a similar polyvalent, cationic radiometal with chemical behavior akin to indium that undergoes radioactive decay by positron emission. Thus, it is proposed that 68Ga-alkylaminoNorBIRT would show similar desirable imaging properties useful in positron-emission tomography or PET. Our previous research has shown these and other radiometals to be effectively incorporated in many DOTA compounds.

Early images which may be obtained 5 minutes post-injection show high concentrations of ¹¹¹In-alkylaminoNorBIRT uptake/retention at the tumor, the result of binding to leukocytes and/or lymphocytes in the cancer tissue. This focal uptake persists at all time points, including images which are obtained 24 hours post-injection. There is prompt and significant radioactivity in the bladder and no focal retention in any other tissues. Biodistribution data following gross dissection of mice at 18 and 24 hrs post-inoculation and tissue harvest correlate well with image-based pharmacokinetic data.

### CONCLUSION

The data evidence that or ⁶⁸Ga- or ¹¹¹In-alkylaminoNorBIRT are highly selective imaging probes for LFA-1 receptor expression, demonstrating high sensitivity and specificity for in vivo SPECT/PET imaging sites of tumor and tumor progression. Other compounds according to the present invention are expected to be effective in much the same way.

1. A method of diagnosing a disease state or condition in a patient comprising administering to said patient an effective amount of at least one compound according to the chemical structure: 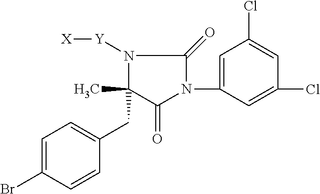
Where Y is a chemical linker which links the nitrogen to a chelate group or tricarbonyl complex X, wherein X incorporates or complexes with a radioisotope, or a pharmaceutically acceptable salt thereof;
measuring the amount of said compound which binds to leukocytes and/or lymphocytes in said tissue in said patient; and comparing the measurement obtained in said measuring step with a standard from uninfected tissue or infected tissue, wherein said measurement obtained from said patient is compared to said standard(s) and said comparison is determined to be indicative of the existence or the absence of said disease state in said tissue.

2. The method according to claim 1 wherein X incorporates a radioisotope selected from the group consisting of ⁹⁰Y, ¹¹¹In, ¹⁷⁷Lu, ²²⁵Ac, ²⁰⁹Bi, ²¹³Bi, ⁶⁷Ga, ⁶⁸Ga, ⁶⁴Cu, ⁶⁷Cu, ⁷¹As, ⁷²As, ⁷⁶As, ⁷⁷As, ⁶⁵Zn, ⁷⁶Br, ⁴⁸V, ⁴⁹V, ²⁰³Pb, ²⁰⁹Pb, ²¹²Pb, ¹⁶⁶Ho, ¹⁵³Pm, ²⁰¹Tl, ¹⁸⁸Re, ¹⁸⁶Re, and ^{99m}Tc and mixtures thereof.

3. The method according to claim 1 or 2 wherein Y is an optionally substituted C₁-C₁₀ hydrocarbyl group.

4. The method according to claim 1 or 2 wherein Y is a —(CH₂)ₙZ— group;
where n is from 1 to 6;
Z is O, NR, N(R)—CH₂CH₂—O—, a keto (C═O) group, a S(O)_{w} group, a phosphonate group or a phosphate group;
R is H or a C₁-C₃ alkyl group;
w is from 0 to 4; and
X is a chelate group in which a radioisotope is incorporated or complexed.

5. The method according to claim 4 wherein Y is a —(CH₂)ₙNH-group, where n is from 2 to 4, preferably 4 and X is a chelate group.

6. The method according to any of claims 1-5 wherein said chelate group is an open-chain polyaminocarboxylate, an AZA macrocycle, a polyaminocarboxylic macrocycles or a polyaminophosphonate macrocycle.

7. The method according to any of claims 1-6 wherein said chelate group is an open-chain polyaminocarboxylate, an AZA macrocycle, a polyaminocarboxylic macrocycles or a polyaminophosphonate macrocycle.

8. The method according to any of claims 1-7 wherein said chelate group is 1,4,7,10-tetraazacyclododecane-1,4,7,10-tetraacetic acid (DOTA).

9. The method according to any of claims 1-8 wherein said radioisotope is ²¹³Bi, ²⁰¹Tl, ¹⁷⁷Lu, ⁶⁸Ga, ⁶⁷Ga or ¹¹¹In.

10. The method according to any of claims 1-9 wherein said radioisotope is ²¹³Bi, ⁶⁸Ga, ⁶⁷Ga or ¹⁷⁷Lu.

11. The method according to any of claims 1-10 wherein said radioisotope is ²¹³Bi, ⁶⁸Ga, ⁶⁷Ga or ¹¹¹In.

12. The method according to any of claim 1-10 wherein said radioisotope is ¹⁷⁷Lu or ¹¹¹In.

13. The method according to claim 1 or 2 wherein said compound is 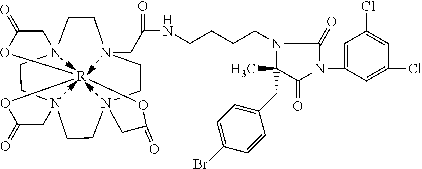
Where R is a radioisotope selected from the group consisting of ⁹⁰Y, ¹¹¹In, ¹⁷⁷Lu, ²²⁵Ac, ²⁰⁹Bi, ²¹³Bi, ⁶⁷Ga, ⁶⁸Ga, ⁶⁴Cu, ⁶⁷Cu, ⁷¹As, ⁷²As, ⁷⁶As, ⁷⁷As, ⁶⁵Zn, ⁷⁶Br, ⁴⁸V, ⁴⁹V, ²⁰³Pb, ²⁰⁹Pb, ²¹²Pb, ¹⁶⁶Ho, ¹⁵³Pm, ²⁰¹Tl, ¹⁸⁸Re, ¹⁸⁶Re, and ^{99m}Tc and mixtures thereof, or a pharmaceutically acceptable salt thereof.

14. The method according to claim 13 wherein R is ²¹³Bi, ¹⁷⁷Lu, ²⁰¹Tl, ⁶⁸Ga, ⁶⁷Ga or ¹¹¹In or a pharmaceutically acceptable salt thereof.

15. The method according to claim 1, 2 or 15 wherein said compound is 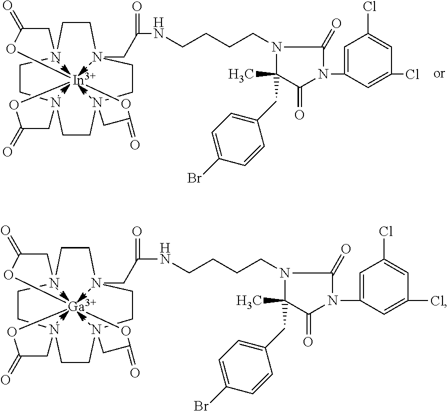
or a pharmaceutically acceptable salt thereof.

16. The method according to claim 13 wherein R is ²¹³Bi, ¹⁷⁷Lu, ²⁰¹Tl, ⁶⁸Ga, ⁶⁷Ga or ¹¹¹In or a pharmaceutically acceptable salt thereof.

17. The method according to any of claims 1-16 wherein said disease state or condition is cancer, neuroinflammation, Alzheimer's disease, Parkinson's disease, Huntington's disease, amyotrophic lateral sclerosis (AML), motor neuron disease (MND), Creutzfeldt-Jacob disease, primary progressive aphasia, progressive supranuclear palsy and other neurodegenerative diseases, chronic pain (including chronic neuropathic pain and central and peripheral neuropathy) or a fatigue disorder.

18. The method according to any of claims 1-17 wherein said disease state or condition is a cancerous tumor.

19. The method according to any of claims 1-17 wherein said disease state or condition is cancer.

20. The method according to claim 19 wherein said cancer is selected from the group consisting of carcinomas (e.g., squamous-cell carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine/endometrial cancer, lung cancer, ovarian cancer, testicular cancer, thyroid cancer, astrocytoma, esophageal cancer, pancreatic cancer, stomach cancer, liver cancer, colon cancer, and melanoma), mixed types of neoplasias, particularly carcinosarcoma and Hodgkin's disease; and tumors of mixed origin, such as Wilms' tumor and teratocarcinomas

21. The method according to any of claims 1-17 wherein said cancer is bowel cancer, breast cancer, prostate cancer, cervical cancer, uterine/endometrial cancer, lung cancer, ovarian cancer, cervical cancer, testicular cancer, thyroid cancer, astrocytoma, esophageal cancer, pancreatic cancer, stomach cancer, liver cancer, colon cancer, and melanoma; mixed types of neoplasias, particularly carcinosarcoma and Hodgkin's disease; and tumors of mixed origin, such as Wilms' tumor and teratocarcinomas.

22. The method according to any of claims 1-17 wherein said cancer is prostate cancer, breast cancer, pancreatic cancer, thyroid cancer, ovarian cancer, lung cancer or liver cancer.

23. A method of monitoring the treatment of a disease state or condition in tissue of a patient in need comprising administering to said patient undergoing a course of treatment for an infectious disease an effective amount of at least one compound according to the chemical structure: 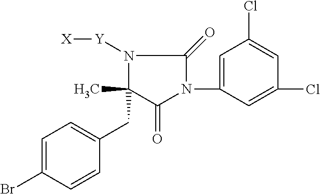
Where Y is a chemical linker which links the nitrogen to a chelate group or tricarbonyl complex X, wherein X incorporates or complexes with a radioisotope, or a pharmaceutically acceptable salt thereof;
measuring the amount of said compound which binds to said tissue in said patient at two different times or more during treatment; and comparing the measurements obtained in said measuring step at said different times with a standard from uninfected tissue and/or infected tissue, wherein said measurements obtained from said patient are compared to said standard(s) and optionally, to each other, such that said comparison is indicative of the progress or absence of progress in the treatment of said disease.

24. The method according to claim 23 wherein X incorporates a radioisotope selected from the group consisting of ⁹⁰Y, ¹¹¹In, ¹⁷⁷Lu, ²²⁵Ac, ²⁰⁹Bi, ²¹³Bi, ⁶⁷Ga, ⁶⁸Ga, ⁶⁴Cu, ⁶⁷Cu, ⁷¹As, ⁷²As, ⁷⁶As, ⁷⁷As, ⁶⁵Zn, ⁷⁶Br, ⁴⁸V, ⁴⁹V, ²⁰³Pb, ²⁰⁹Pb, ²¹²Pb, ¹⁶⁶Ho, ¹⁵³Pm, ²⁰¹Tl, ¹⁸⁸Re, ¹⁸⁶Re, and ^{99m}Tc and mixtures thereof.

25. The method according to claim 23 or 24 wherein Y is an optionally substituted C₁-C₁₀ hydrocarbyl group.

26. The method according to claim 23 or 24 wherein Y is a —(CH₂)ₙZ-group;
where n is from 1 to 6;
Z is O, NR, N(R)—CH₂CH₂—O—, a keto (C═O) group, a S(O)_{w} group, at phosphonate group or a phosphate group;
R is H or a C₁-C₃ alkyl group;
w is from 0 to 4; and
is a dictate group in which a radioisotope is incorporated complexed.

27. The method according to claim 26 wherein Y is a —(CH₂)ₙNH— group, where n is from 2 to 4, preferably 4 and X is a chelate group.

28. The method according to any of claims 23-27 wherein said chelate group is an open-chain polyaminocarboxylate, an AZA macrocycle, a polyaminocarboxylic macrocycles or a polyaminophosphonate macrocycle.

29. The method according to any of claims 23-28 wherein said chelate group is an open-chain polyaminocarboxylate, an AZA macrocycle, a polyaminocarboxylic macrocycles or a polyaminophosphonate macrocycle.

30. The method according to any of claims 23-29 wherein said chelate group is 1,4,7,10-tetraazacyclododecane-1,4,7,10-tetraacetic acid (DOTA).

31. The method according to any of claims 23-30 wherein said radioisotope is ²¹³Bi, ²⁰¹Tl, ¹⁷⁷Lu, ⁶⁸Ga, ⁶⁷Ga or ¹¹In or a pharmaceutically acceptable salt thereof.

32. The method according to any of claims 23-31 wherein said radioisotope is ²¹³Bi, ⁶⁸Ga, ⁶⁷Ga or ¹⁷⁷Lu or a pharmaceutically acceptable salt thereof.

3. The method according to any of claims 23-31 wherein said radioisotope is ²¹³Bi, ⁶⁸Ga, ⁶⁷Ga or ¹¹¹In or a pharmaceutically acceptable salt thereof.

34. The method according to an of claim 23-31 wherein said radioisotope is ¹⁷⁷Lu, ⁶⁸Ga, ⁶⁷Ga or a pharmaceutically acceptable salt thereof.

35. The method according to any of claim 23 or 24 wherein said compound is 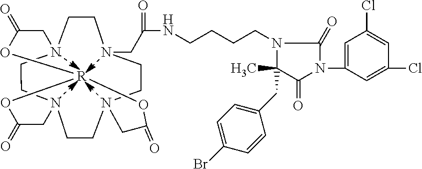
Where R is a radioisotope selected from the group consisting of ⁹⁰Y, ¹¹¹In, ¹⁷⁷Lu, ²²⁵Ac, ²⁰⁹Bi, ²¹³Bi, ⁶⁷Ga, ⁶⁸Ga, ⁶⁴Cu, ⁶⁷Cu, ⁷¹As, ⁷²As, ⁷⁶As, ⁷⁷As, ⁶⁵Zn, ⁷⁶Br, ⁴⁸V, ⁴⁹V, ²⁰³Pb, ²⁰⁹Pb, ²¹²Pb, ¹⁶⁶Ho, ¹⁵³Pm, ²⁰¹Tl, ¹⁸⁸Re, ¹⁸⁶Re, and ^{99m}Tc and mixtures thereof.

36. The method according to claim 35 wherein Ri is ²¹³Bi, ¹⁷⁷Lu, ⁶⁸Ga, ⁶⁷Ga or ¹¹¹In or a pharmaceutically acceptable salt thereof.

37. The method according to claim 35 wherein Ri is ²¹³Bi, ⁶⁸Ga, ⁶⁷Ga or ¹¹¹In or a pharmaceutically acceptable salt thereof.

38. The method according to claim 35 wherein Ri is ²¹³Bi, ¹⁷⁷Lu, ²⁰¹Tl, ⁶⁸Ga, ⁶⁷Ga or ¹¹¹In or a pharmaceutically acceptable salt thereof.

39. The method according to claim 23 or 24 wherein said compound is 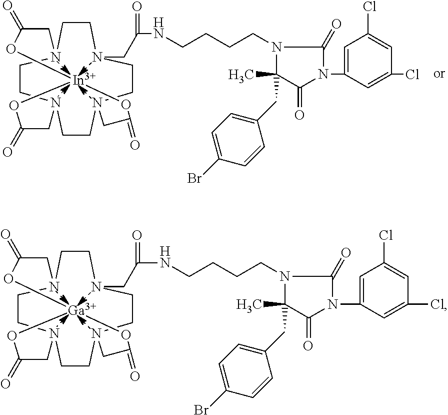
or a pharmaceutically acceptable salt thereof.

40. The method according to any of claims 23-39 wherein said disease state or condition is cancer, neuroinflammation, Alzheimer's disease, Parkinson's disease, Huntington's disease, amyotrophic lateral sclerosis (AML), motor neuron disease (MND), Creutzfeldt-Jacob disease, primary progressive aphasia, progressive supranuclear palsy and other neurodegenerative diseases, chronic pain (including chronic neuropathic pain and central and peripheral neuropathy) or a fatigue disorder.

41. The method according to any of claims 23-39 wherein said disease state or condition is a cancerous tumor.

42. The method according to any of claims 23-40 wherein said disease state or condition is cancer.

43. The method according to claim 41 or 42 wherein said treatment of said cancer is by immunotherapy.

44. The method according to claim 43 wherein said immunotherapy is chimeric antigen receptor T-cell (CART) therapy, T-cell receptor therapy (TRT therapy), tumor-infiltrating lymphocytes (TIL therapy), monoclonal antibodies, immune checkpoint inhibitors, cancer vaccines or general immunotherapy (e.g., interleukins, interferons, colony stimulating factors and as which boost the immune system such as imiquimod (Zyclara), lenalidomide (Revlimid), pomalidomide (Pomalyst), and thalidomide).

45. The method according to any of claim 40 or 42-44 wherein said cancer is selected from the group consisting of carcinomas (e.g., squamous-cell carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas): germ-line tumors (e.g., bowel cancer, breast cancer, prostate cancer, cervical cancer, uterine/endometrial cancer, lung cancer, ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas.

46. The method according to claim 40 or 42-44 wherein said cancer is bowel cancer, breast cancer, prostate, cancer, cervical cancer, uterine/endometrial cancer, lung cancer, ovarian cancer, cervical cancer, testicular cancer, thyroid cancer, astrocytoma, esophageal cancer, pancreatic cancer, stomach cancer, liver cancer, colon cancer, and melanoma; mixed types of neoplasias, particularly carcinosarcoma and Hodgkin's disease; and rumors of mixed origin, such as Wilms' tumor and teratocarcinomas.

47. The method according to any of claim 40 or 42-46 wherein said cancer is prostate cancer, breast cancer, pancreatic cancer, thyroid cancer, ovarian cancer, lung cancer or liver cancer.

48. The method according to any of claims 23-47 wherein said therapy is modified after said monitoring.

49. A pharmaceutical composition comprising an effective amount of at least one compound according to the chemical structure: 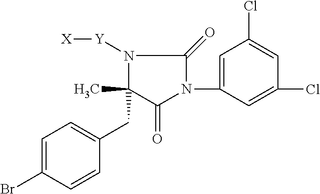
Where V is a chemical linker which links the nitrogen to a dictate group or tricarbonyl complex X, wherein X incorporates or complexes with a radioisotope, or a pharmaceutically acceptable salt thereof in combination with a pharmaceutically acceptable carrier, additive or excipient and optionally in further combination with an effective amount of an anticancer agent.

50. The composition according to claim 49 wherein X incorporates a radioisotope selected from the group consisting of ⁹⁰Y, ¹¹¹In, ¹⁷⁷Lu, ²²⁵Ac, ²⁰⁹Bi, ²¹³Bi, ⁶⁷Ga, ⁶⁸Ga, ⁶⁴Cu, ⁶⁷Cu, ⁷¹As, ⁷²As, ⁷⁶As, ⁷⁷As, ⁶⁵Zn, ⁷⁶Br, ⁴⁸V, ⁴⁹V, ²⁰³Pb, ²⁰⁹Pb, ²¹²Pb, ¹⁶⁶Ho, ¹⁵³Pm, ²⁰¹Tl, ¹⁸⁸Re, ¹⁸⁶Re, and ^{99m}Tc and mixtures thereof.

51. The composition according to claim 49 or 50 wherein Y is an optionally substituted C₁-C₁₀ hydrocarbyl group.

52. The composition according to any of claims 49-51 wherein Y is a —(CH₂)ₙZ-group;
where n is from 1 to 6;
Z is O, NR, N(R)—CH₂CH₂—O , a keto (C═O) group, a S(O)_{w} group, a phosphonate group or a phosphate group;
R is H or a C₁- C₁₀ alkyl group;
w is from 0 to 4; and
X is a chelate group in which a radioisotope is incorporated or complexed.

53. The composition according to claim 49 wherein Y is a —(CH—)ₙNH-group, where n is from 2 to 4, preferably 4 and X is a chelate group.

54. The composition according to any of claims 49-53 wherein said chelate group is an open-chain polyaminocarboxylate, an AZA macrocycle, a polyaminocarboxylic macrocycle or a polyaminophosphonate macrocycle.

55. The composition method according to any of claims 49-53 wherein said chelate group is an open-chain polyaminocarboxylate, an AZA macrocycle or a polyaminocarboxylic macrocycle.

56. The composition according to any of claims 49-55 wherein said chelate group is 1,4,7,10-tetraazacyclododecane-1,4,7,10-tetraacetic acid (DOTA).

57. The composition according to any of claims 49-56 wherein said radioisotope is ²¹³Bi, ²⁰¹Tl, ¹⁷⁷Lu, ⁶⁷Ga, ⁶⁸Ga or ¹¹¹In.

58. The composition according to any of claims 47-55 wherein said radioisotope is ²¹³Bi, ⁶⁷Ga, ⁶⁸Ga or ¹⁷⁷Lu.

59. The composition according to any of claims 47-55 wherein said radioisotope is ²¹³Bi, ⁶⁷Ga, ⁶⁸Ga or ¹¹¹In.

60. The composition according to any of claims 47-55 wherein said radioisotope is ¹⁷⁷Lu, ⁶⁷Ga, ⁶⁸Ga or ¹¹¹In.

61. The composition according to claim 47 or 48 wherein said compound is 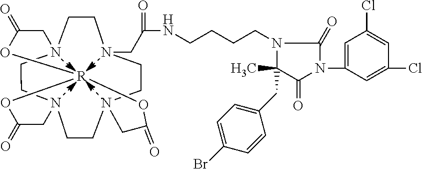
Where R is a radioisotope selected from the group consisting of ⁹⁰Y, ¹¹¹In, ¹⁷⁷Lu, ²²⁵Ac, ²⁰⁹Bi, ²¹³Bi, ⁶⁷Ga, ⁶⁸Ga, ⁶⁴Cu, ⁶⁷Cu, ⁷¹As, ⁷²As, ⁷⁶As, ⁷⁷As, ⁶⁵Zn, ⁷⁶Br, ⁴⁸V, ⁴⁹V, ²⁰³Pb, ²⁰⁹Pb, ²¹²Pb, ¹⁶⁶Ho, ¹⁵³Pm, ²⁰¹Tl, ¹⁸⁸Re, ¹⁸⁶Re, and ^{99m}Tc and mixtures thereof.

63. The composition according to claim 47, 48, 59 or 60 wherein said compound is 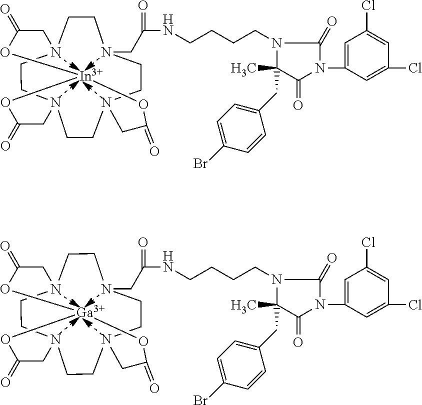
or a pharmaceutically acceptable salt thereof.

64. The composition according to any of claims 47-63 wherein said composition includes an anticancer agent which is at least one agent selected from the group consisting of antimetabolites, inhibitors of topoisomerase I and II, alkylating agents and microtubule inhibitors.

65. The composition according to any of claims 47-63 which includes an anticancer agent and wherein said anticancer agent is at least one agent selected from the group consisting of everolimus, trabectedin, abraxane, TLK 286, AV-299, DN-101, pazopanib, GSK690693, RTA 744, ON0910.Na, AZD6244 (ARRY-142886), AMN-107, TKI-258, GSK461364, AZD1152, enzastaurin, vandetanib, ARQ-197, MK-0457, MLN8054, PHA-739358, R-763, AT-9263, a FLT-3 inhibitor, a VEGFR inhibitor, an EGFR K inhibitor, an aurora kinase inhibitor, a PIK-1 modulator, a Bel-2 inhibitor, an HDAC inhibitor, a c-M ET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decinanib, panitumumab, amrubicin, oregovomab, Lep-etu, nolatrexed, azd2171, batabulin, ofatumumab (Arzerra) zanolimumab, edotecarin, tetrandrine, rubitecan, tesmilifene, oblimersen, ticilimumab, ipilimumab, gossypol, Bio 111 , 131-I-TM-601, ALT-110, BIO 140, CC 8490, cilengitide, gimatecan IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, irinotecan, liposomal doxorubicin, 5′-deoxy-5-fluorouridine, vincristine, temozolomide, ZK-304709, seliciclib PD0325901, AZD-6244, capecitabine, L-Glutamic acid, N-[4-[2-(2-amino-4,7-dihydro-4-oxo-1 H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258,); 3-[5-(methylsulfonylpiperadinemethyl)-indolyl]-quinolone, vatalanib, AG-013736, AVE-0005, the acetate salt of [D-Ser(Bu t) 6, Azgly 10] (pyro-Glu-His-Trp-Ser-Tyr-D-Ser(Bu t)-Leu-Arg-Pro- Azgly-NH₂ acetate [C₅₉H₈₄N₁₈Oi₄—(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitax, EKB-569, PKI-66, GW-572016, Ionafarnib, BMS-214662, tipifarnib; amifostine, NVP-LAQ824, suberoyl analide hydroxamic acid, valproic acid, trichostatin A, FK-228, SU11248, soratenib, KRN951, aminoglutethimide, arnasacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topetecan, razoxin, marimastat, COL-3, neovastat, BMS-275291, squalamine, endostatin, SU5416, SU6668, EMD121974, interleukin-12, IM862 angiestatin, vitaxin, droloxifene, idoxyfene, spironolactone, finasteride, cimitidine, trastuzumab, denileukin diftitox, gefitinib, bortezimib, paclitaxel, irinotecan, topotecan, doxorubicin, docetaxel, vinorelbine, bevacizumab (monoclonal antibody) and erbitux, cremophor-free paclitaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, PTK787ZK222584, VX-745, PD 184352, rapamycin, 40-O-(2-hydroxyethyl)-rapamycin, temsirolimus, AP-23573, RAD001, ABT-578, BC-210, LY94002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa, ipilimumab, pembrolizumab, nivolumab, alemtuzumab, atezolizumab, ofatumumab, rituximab and mixtures thereof.

66. A method of diagnosing the existence of a metastatic cancer of a patient comprising administering to said patient an effective amount of at least one compound according to the chemical structure: 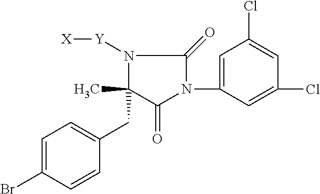
Where Y is a chemical linker which links the nitrogen to a chelate group or tricarbonyl complex X, wherein X incorporates or complexes with a radioisotope, or a pharmaceutically acceptable salt thereof.
measuring the amount of said compound which binds to said tissue in said patient; and
comparing the measurement obtained in said measuring step with a standard from uninfected tissue or metastatic tissue, wherein said measurement obtained from said patient is compared to said standard(s) and said comparison is determined to be indicative of the existence or the absence of metastatic cancer in said tissue.

67. The method according to claim 66 wherein after said comparison is determined to be indicative of the existence of metastatic cancer, therapy is initiated.

68. A method of treating and monitoring cancer in a patient comprising administering to said patient an effective amount of at least one composition according to an of claims 47-65 to said patient.

69. The method according to claim 68 wherein said cancer is selected from the group consisting of carcinomas (e.g., squamous-cell carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas;
myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine/endometrial cancer, lung cancer, ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas.

70. The method according to claim 68 wherein said cancer is bowel cancer, breast cancer, prostate cancer, cervical cancer, uterine/endometrial cancer, lung cancer, ovarian cancer, cervical cancer, testicular cancer, thyroid cancer, astrocytoma, esophageal cancer, pancreatic cancer, stomach cancer, liver cancer, colon cancer, and melanoma; mixed types of neoplasias, particularly carcinosarcoma and Hodgkin's disease; and tumors of mixed origin, such as Wilms' tumor and teratocarcinomas.

71. The method according to claim 68 wherein said cancer is prostate cancer, breast cancer, pancreatic cancer, thyroid cancer, ovarian cancer, lung cancer or liver cancer.

72. The method according to claim any of claims 68-71 wherein said treating of cancer is or includes immunotherapy.

73. The method according to claim 72 wherein said immunotherapy is chimeric antigen receptor T-cell (CART) therapy, T-cell receptor therapy (TRT therapy), tumor-infiltrating lymphocytes (TIL therapy), monoclonal antibodies, immune checkpoint inhibitors, cancer vaccines or general immunotherapy (e.g., interleukins, interferons, colony stimulating factors and agents which boost the immune system such as imiquimod (Zyclara), lenalidomide Revlimid), pomalidomide (Pomalyst), and thalidomide).

74. A method of treating and monitoring cancer in a patient in need thereof comprising administering to said patient an effective amount of at least one composition according to any of claims 49-65 to said patient and measuring the amount of compound contained in said composition which binds to leukocytes and/or lymphocytes in cancerous tissue in said patient at two different times or more during treatment; and comparing the measurements obtained in said measuring step at said different times with a standard from uninfected tissue and/or infected tissue, wherein said measurements obtained from said patient are compared to said standard(s) and optionally, to each other, such that said comparison is indicative of the progress or absence of progress in the treatment of said infectious disease.

75. The method according to claim 74 wherein said cancer is said cancer is selected from the group consisting of carcinomas (e.g., squamous-cell carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas;
myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine/endometrial cancer, lung cancer, ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas

76. The method according to claim 74 wherein said cancer is bowel cancer, breast cancer, prostate cancer, cervical cancer, uterine/endometrial cancer, lung cancer, ovarian cancer, cervical cancer, testicular cancer, thyroid cancer, astrocytoma, esophageal cancer, pancreatic cancer, stomach cancer, liver cancer, colon cancer, and melanoma; mixed types of neoplasias, particularly carcinosarcoma and Hodgkin's disease; and tumors of mixed origin, such as Wilms' rumor and teratocarcinomas.

77. The method according to claim 74 wherein said cancer is prostate cancer, breast cancer, pancreatic cancer, thyroid cancer, ovarian cancer, lung cancer or liver cancer.

78. The method according to any of claims 74-77 wherein said treatment of said cancer is or includes immunotherapy.

79. The method according to claim 78 wherein said immunotherapy is chimeric antigen receptor T-cell (CART) therapy, T-cell receptor therapy (TRI therapy), tumor-infiltrating lymphocytes (TIL therapy), monoclonal antibodies, immune checkpoint inhibitors, cancer vaccines or general immunotherapy (e.g., interleukins, interferons, colony stimulating factors and agents which boost the immune system such as imiquimod (Zyclara), lenalidomide (Revlimid), pomalidomide (Pomalyst), and thalidomide).

80. Use of a composition according to any of claims 49-65 in the manufacture of a medicament for the monitoring and treatment of cancer in a patient.

71. Use of a composition as set forth in any of claims 49-63 in the manufacture of a medicament for the diagnosis of the existence of cancer in a patient.

72. Use of a composition as set forth in any of claims 49-63 in the manufacture of a medicament for monitoring the progress of therapy in treating cancer in a patient.
